(19) United States
(12) Patent Application Publication
(10) Pub. No.: US 2019/0307901 A1
(43) Pub. Date: Oct. 10, 2019

(54) METHOD FOR ENHANCED NUCLEIC ACID TRANSFECTION USING A PEPTIDE

(71) Applicant: Yuan Zhang, Saunderstown, RI (US)

(72) Inventor: Yuan Zhang, Saunderstown, RI (US)

(21) Appl. No.: 15/979,874

(22) Filed: May 15, 2018

Related U.S. Application Data
(60) Provisional application No. 62/655,012, filed on Apr. 9, 2018.

(51) Int. Cl. A61K 48/00; A61K 9/127

(52) U.S. Cl. CPC A61K 48/0066; A61K 9/1272

(57) ABSTRACT

A method for genetic transfection of mammalian cells is disclosed using a novel nanoparticle. The method comprises of mixing cationic peptides to nucleic acids, and then subsequently incorporating this mixture into a liposome with surface modification for transfection purposes and other medical application. With various types of cationic peptides envisioned, the method can be used for developing a nanoparticle comprising of either anionic or cationic liposomes, and for incorporating any type of nucleic acid. The medical application of this technology includes, but not limited to, gene therapy and nucleic acid based vaccination against a broad range of diseases, such as cancer and infectious disease.

### FIELD OF THE INVENTION

The present invention generally relates to method of nucleic acid transfection using a novel nanoparticle. Specifically, the method entails combining nucleic acid with peptides prior to incorporating such combination within a novel nanoparticle for transfection. The resulting effect is an increase in transfection efficiency of desired nucleic acid in mammalian cells.

### CROSS-REFERENCE TO RELATED APPLICATIONS

This application claims the benefit of the provisional application entitled METHOD FOR ENHANCED DELIVERY OF GENE BASED THERAPY AND VACCINATION USING ELECTROPORATION with U.S. Application No. 62/655,012 filed on Apr. 9, 2018.

### BACKGROUND OF THE INVENTION

Transfection is a potent analytical tool that enables the study of nucleic acids within a cell's own machinery. It is the process of inserting foreign genetic material (e.g. DNA and RNA) into mammalian cells to evaluate gene expression and gene inhibition for use for a variety of applications, including gene therapy while minimizing unintended effects such as cytotoxicity, off-target gene perturbation, or cell death.

The insertion of genetic materials into cells can be induced by various biological, chemical and physical means depending on a multitude of factors and desired goals. For examples, the type of nucleic acid being transfected, the cell line, and the nature of transfection (stable v. transient) should be considered when deciding the best transfection method to employ.

One method of transfection involves the use of nanoparticles as carriers for nucleic acid. Nanoparticles are small (generally under 200 nanometers) objects that are either naturally or artificially produced. Despite their small size, they have a comparatively larger surface for cellular adhesion compared to other particles for transfection, allowing them to cross the cell membrane into the cell with significant efficiency. Further, in addition to their enhanced propensity for transport into a cell, nanoparticles shield the incorporated nucleic acid from degradation by nucleases.

In general, nanoparticle transfection is preferred over other transfection methods due to its sustained release characteristics and enhanced safety profile and biocompatibility over virus-mediated gene transfer. Nanoparticles efficiently enter cells by exploiting the endocytosis pathway, followed by the entrapment in the acidic endosomal and lysosomal compartments for degradation. To prevent enzymatic degradation intracellularly, certain endosome-disrupting agents or rationally designed biomaterials (i.e. lipids, polymers) are usually incorporated into the nanoparticle carriers in order to promote the release of therapeutic cargos (i.e. nucleic acids) entrapped in nanoparticles from endosomes/lysosomes to cytoplasm in order to exert their biological functions.

As technology has advanced, nanoparticle transfection has shifted from predominantly focusing on the transfer of DNA to RNA. There are certain advantages with the use of RNA, especially when the desired downstream application is to utilize genetic targets for vaccination. First, there is little risk that the RNA may integrate into the host genome. Second, in order for transfection of the DNA to be successful, the DNA must not only cross the cytoplasmic membrane, but the nuclear membrane as well; in contrast, RNA needs only to cross the cytoplasmic membrane to induce the desire effect. Lastly, transfection of RNA can create desired downstream effects in even slow or non-dividing cells.

RNA replicons are one such type of nucleic acid that has been posed for transfection through the employment of nanoparticles. RNA replicons (self-amplifying RNA) are derived from either positive- or negative-strand RNA viruses. RNA replicons contain the genes encoding the RNA virus replication machinery (non-structure proteins), but lack the genes encoding the viral structural proteins required to make an infectious virus particle. Rather, the structural protein genes are replaced with genes encoding proteins of interest for various medical applications. Since at least one gene encoding essential structural protein has been deleted, they are regarded as “disabled” viruses, and they are unable to produce infectious progeny.

Despite the benefit of RNA transfection, the intracellular delivery and transfection of RNA replicons remains a significant challenge for gene therapy. Thus there is a need in the arts for improving the transfection efficiency of RNA replicon using nanoparticles as non-viral delivery systems.

### SUMMARY OF THE INVENTION

In one aspect, the present invention is directed to a method of administering genetic materials into eukaryotic cells comprising of the steps of preparing a condensed core, there the condensed core comprises of a nucleic acids and peptides, combining the condensed core with liposomes to form a nanoparticle, and transfecting the mixture of condensed core and liposomes into eukaryotic cells. In the preferred embodiment, the peptides are water soluble.

In another aspect of the claimed invention, the method employs cationic peptides. In one embodiment, the peptide comprises of a peptide sequence containing four or more arginine residues. Another cationic peptide that can be utilized in accordance with the current invention include (but are not limited to) SIGMA's protamine peptide. In alternate embodiments, the claimed invention employs anionic peptides. Yet, in further embodiments, the claimed invention employs both cationic and anion peptides.

According to embodiments of the current invention, cationic liposomes are employed in synthesizing the nanoparticles. Specifically, the nanoparticles are formed using liposomes comprised of cationic lipids. In one embodiment, the cationic lipids are comprised of at least one amine group. In alternate embodiments of the claimed invention, the cationic liposomes are comprised of a 1:1 molar ratio of 1,2-dioleoyl-3-trimethylammonium-propane (DOTAP) and cholesterol.

According to embodiments of the current invention comprising of anionic liposomes, such liposomes are further comprised of a 2:1:1 molar ratio of 1 2-dioleoyl-sn-glycero-3-phosphate (DOPA), 1 2-dioleoyl-sn-glycero-3-phosphoethanolamine (DOPE), and cholesterol.

According to embodiments of the current invention, the nanoparticle comprising of liposomes and a condensed core (which itself comprises of a nucleic acid and peptides) is coated with hydrophilic phospholipids. In some embodiments, the hydrophilic phospholipids in a density less than 20% of the total lipids of the final nanoparticles.

According to embodiments of the current invention, the condensed core is either positively charged, neutrally charged, or negatively charged.

According to embodiments of the current invention, the nucleic acid to be transfected comprises of small nucleic acids that weigh less than 100,000 Da.

According to embodiments of the current invention, the condensed core is further combined with high molecular weight carrier molecules.

According to embodiments of the current invention, the condensed core is combined with polysaccharides or high molecular weight DNA.

According to embodiments of the current invention, the transfection method can be administered in vitro, in vivo, and ex vivo.

According to another embodiment of the current invention, a method for delivering genetic materials into eukaryotic cells comprising of preparing a condensed core comprising of RNA replicon and cationic peptides, combining the condensed core with cationic liposomes comprised of cationic lipids, adding 1,2-distearoyl-sn-glycero-3-phosphoethanolamine-N-[methoxy(polyethylene glycol)-2000 (DSPE-PEG) or other phospholipids to the condensed core and liposomes, and transfecting this mixture of condensed core, liposomes, and DSPE-PEG into eukaryotic cells. In some embodiments, the cationic liposomes are comprised of both cationic lipids and neutral lipids.

According to embodiments of the alternative invention, the nanoparticle comprising of condensed core, liposomes, and DSPE-PEG is followed by surface modifications to improve its physiochemical properties, cell targeting specificities and pharmacokinetic (PK) profiles.

According to embodiments of the alternative invention, for LPR entrapping RNA replicon, the density of DSPE-PEG is less than 5% of the total lipids.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1A-1C show comparisons of transfection efficiency by concentrations of RNA replicon formulated in Lipid nanoparticle with no peptide (“LNP”) (FIG. 1A) or LPR (FIG. 1A-1C) as measured by a luciferase assay in various cell types.

FIG. 2 shows the transfection efficiency using two different cationic peptide sequences as measured by a luciferase assay. CR8C represents peptide sequence CRRRRRRRRC.

FIG. 3 shows how the transfection efficiency of RNA replicon is impacted by increasing the amount of DSPE-PEG anchored on LPRs.

FIG. 4A-6B shows confocal microscope captured imagery of cells treated with LPR in accordance with the claimed method. Blue color is nuclei stained with DAPI. Green color is endosome/lysosome stained with Lysotracker Green DND-26. Red color is replicon labeled with Cy3 dye.

### DETAILED DESCRIPTION OF THE INVENTION

Abbreviations used through this description have the following meaning:
DAPI: 2-(4-amidinophenyl)-1H-indole-6-carboxamidine
DOPA: 1 2-dioleoyl-sn-glycero-3-phosphate
DOPE: 1 2-dioleoyl-sn-glycero-3-phosphoethanolamine
DOTAP: 1,2-dioleoyl-3-trimethylammonium-propane
DSPC: 1,2-diastearoyl-sn-glycero-3-phosphocholine
DSPE-PEG: 1,2-distearoyl-sn-glycero-3-phosphoethanolamine-N-[methoxy(polyethylene glycol)-2000

The invention is a method for delivering nucleic acid to eukaryotic cells using novel lipid-based nanoparticles containing peptide. While the typical and established high-level protocols for nanoparticle transfection are not altered, the claimed invention fine-tunes such protocols by changing the environment in which the exogenous nucleic acid resides and using a novel nanoparticle. In brief, the method entails combining the desired nucleic acid (to be transfected) with peptides (such combination hereinafter referred to as a “Condensed Core”), and then subsequently adding liposomes to the Condensed Core to form the nanoparticle for transfection. In the preferred embodiment, the Condensed Core comprises of cationic peptides and RNA replicon (hereinafter “Replicon Condensed Core”). The Condensed Core is then combined with liposomes to form a novel lipid-based peptide nanoparticle (hereinafter “LPR” or “nanoparticle”) that is subsequently used in transfection procedures.

The nucleic acid in the Condensed Core is negatively charged due to the phosphate groups forming the polar backbones. Accordingly, in the preferred embodiment, the addition of cationic peptides generates stabilized electrostatic interactions between the positively charged cationic peptides and the negatively changed nucleic acid that facilitates transfection. Despite such stabilization, the Condensed Core in the preferred embodiment is nonetheless charged such that it may exhibit a net positive charge or net negative charge depending on the ratio of cationic peptide and nucleic acid. The net charge of the Condensed Core determines the proper liposome to use for transfection. For example, a negatively charged Condensed Core is combined with a cationic liposome for transfection purposes, while a positively charged Condensed Core is combined with an anionic liposome for transfection purposes.

The novelty of the current claimed invention lies in the dramatic increase in transfection efficiency of large-size nucleic acids in mammalian cells by the addition of peptides and the use of a nanoparticle specific for the current purposes. Data utilizing the preferred method employing the Replicon Condensed Core and the LPR shows more than a 100× increase in transfection efficacy as determined by a luciferase assay. Previously, cationic oligopeptides have been known to be used as nonviral delivery vectors due to their excellent biocompatibility and nontoxicity to allow for efficient delivery of exogenous nucleic acids. Nonetheless transfection efficiency has been low due to their poor stability, especially in large RNA (˜10 kb). To remedy the problem, rather than use cationic peptides as the delivery vectors, the cationic peptides are incorporated within novel nanoparticles. This allows for the nanoparticles to be used as the delivery vector while housing the Condensed Core.

In the preferred embodiment, the Condensed Core is comprised of the cationic peptide protamine sulfate (SIGMA Catalog #P4020) (hereinafter “Protamine”). Alternative cationic peptides suitable for the claimed method include peptide sequences rich in lysine, arginine such as CRRRRRRRRC (hereinafter referred to as “CR8C”), and histidine, such as CHHHHC. As will be discussed below, the result of using a cationic peptide incorporated within a nanoparticle along with large nucleic acid (i.e. RNA replicon) is a significant increase in transfection efficiency. In alternate embodiments, anionic peptides can also be used, in lieu of or together with other cationic peptides.

In one embodiment of the current invention, cationic lipids are used to formulate the liposomes. Such cationic lipids contain one or more primary, secondary and/or tertiary amine groups, including but not limited to DOTAP, dimethyldioctadecylammonium (DDAB), 1,2-di-O-octadecenyl-3-trimethylammonium propane (DOTMA), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (EDOPC), D-erythro-2-N-[6′-(1″-pyridinium)-hexanoyl]-sphingosine, 1,2-dipalmitoyl-sn-glycero-3-[phosphor-rac-(3-lysyl(1-glycerol))], 1,2-dioleoyl-sn-glycero-3-[phosphor-rac-(3-lysyl(1-glycerol))], as well as other cationic lipids.

In another embodiment, the liposomes are anionic liposomes in that they contain phosphate, sulfate and/or carboxyl groups, including but not limited to DOPA, 1,2-diphytanoyl-sn-glycero-3-phospho-(1′-rac-glycerol), 1,2-diphytanoyl-sn-glycero-3-phospho-L-serine, 1,2-diphytanoyl-sn-glycero-3-phosphate, phytanoyl coenzyme A, 1,2-dioleoyl-sn-glycero-3-phospho-L-serine (DOPS), 1,2-dioleoyl-sn-glycero-3-(cytidine diphosphate), dioctanoylglycerol pyrophosphate, dioleoylglycerol pyrophosphate, as well as other anionic lipids.

Yet, in other alternate embodiments, the liposomes, whether cationic or anionic, are further comprised of fusogenic lipids. The fusogenic lipids are typically neutral lipids that promote the formation of lipid structures in order to favor the cellular uptake and transfection efficiency of synthetic particles. In other words, fusogenic lipids promote the organization of lipids into stable bilayers to form liposomes. Examples of fusogenic lipids that can be used to formulate the liposomes include, but are not limited to, cholesterol, DOPE, 1,2-dioleoyl-sn-glycero-3-phosphocholine (DOPC), 1,2-bis (10,12-tricosadiynoyl)-sn-glycero-3-phosphocholine (DC(8,9)PC), 1-palmitoyl-2-(9′-oxo-nonanoyl)-sn-glycero-3-phosphocholine, 1,2-dipalmitoyl-sn-glycero-3-O-4′-(N,N,N-trimethyl)-homoserine (DGTS), 1,2-dipalmitoyl-sn-glycero-3-O-4′-[N,N,N-trimethyl(d9)]-homoserine (DGTS-d9), (palmitoyloxy) octadecanoic acid (PAHSA), [((13,13,14,14,15,15,16,16,16-d9)palmitoyl)hydroxyl]-stearic acid (PAHSA-d9), urea-ceramide, 1-hexadecyl-2-(9Z-octadecenoyl)-sn-glycero-3-phosphoethanolamine, 1-myristoyl-2-(4nitrophenylsuccinyl)-sn-glycero-3-phosphocholine, 1-O-hexadecyl-2-oleoyl-sn-glycero-3-phosphocholine, 1-O-hexadecyl-2-(eicosatrienoyl)-sn-glycero-3-phosphocholine, 1-oleoyl-N-heptadecanoyl-D-erythro-sphingosine, N-lauroyl-1-deoxysphingosine, N-palmitoyl-1-deoxysphingosine, N-palmitoyl-1-desoxymethylsphingosine, N-lauroyl-1-desoxymethylsphingosine, N-nervonoyl-1-desoxymethylsphinganine, N-nervonoyl-1-deoxysphingosine, N-(1-adamantaneacetyl)glucosylceramide, 1-desoxymethylsphingosine, 1-deoxysphingosine, (R)-3-(3-tetradecylureido)-4-(trimethylammonio) butanoate.

In some embodiments of the current invention, the LPRs are coated with hydrophilic phospholipids and/or their derivatives. Such hydrophilic phospholipids comprise of a hydrophilic head segment and hydrophobic tail segment. Examples of hydrophilic phospholipids include but are not limited to DSPE-PEG, 1,2-dimyristoyl-rac-glycero-3-methylpolyoxyethylene (DMG-PEG), N-(methylpolyoxyethylene oxycarbonyl)-1,2-dipalmitoyl-sn-glycero-3-phosphoethanolamine (DPPE-PEG), N-(methylpolyoxyethylene oxycarbonyl)-1,2-dimyristoyl-sn-glycero-3-phosphoethanolamine (DMPE-PEG), 1,2-distearoyl-rac-glycero-3-methylpolyoxyethylene (DS G-PEG), 1,2-dipalmitoyl-rac-glycero-3-methylpolyoxyethylene (DPG-PEG), and 1,2-dioleoyl-rac-glycerol methoxypolyethylene glycol (DOG-PEG). The amount of hydrophilic phospholipids used to coat the LPR depends on the total lipids comprising the LPR. In general, the amount of hydrophilic phospholipids used to coat the nanoparticles comprises of approximately 1%-20% of the total lipids used in the formulation of the LPR. For embodiments consisting of the Replicon Condensed Core, it is preferable for the hydrophilic phospholipids to be less than 5% of the total lipids used in the LPR.

In one embodiment, DSPE-PEG is used to coat the surface of the LPR. DSPE-PEG is a biocompatible, biodegradable and amphiphilic phospholipid-polymer conjugate. When used as a coating, DSPE-PEG renders the nanoparticle water-dispersible, prevents aggregation, reduces non-specific absorption in biological systems, and provides other functionalities. The size, charge, hydrophilicity and flexibility of the DSPE-PEG molecules are critical mediators for the in vitro and in vivo performance of nanoparticles. In the current invention, the method of RNA replicon transfection involves a novel nanoparticle that is coated with 1%-5% DSPE-PEG relative to the amount of total lipids in the nanoparticle. In the preferred embodiment, the DSPE-PEG is 2% of the total lipid concentration.

Schematically, the Condensed Core is the interior part of the liposome while the lipid layer coats the outside of the Condensed Core resulting in the formation of the LPR. If DSPE-PEG (or some other hydrophilic phospholipid) is added, the hydrophobic component, e.g. the DSPE, will insert itself into the exterior lipid layer of the LPR while the hydrophilic component, e.g. the PEG stretches to stabilize the LPR and suspend it in solution.

In one aspect, the present invention is directed to a method for delivering an RNA replicon machinery. The RNA replicon encodes non-structure proteins for replication capability and the gene of interest for the expression of the desired protein. Specifically the RNA replicon is combined with a peptide to form a Condensed Core. The Condensed Core is subsequently wrapped around with a liposome to form a lipid nanoparticle. This is followed by surface modifications to improve its physiochemical properties, cell targeting specificities and pharmacokinetic (PK) profiles.

Further embodiments of the current invention include surface modifications of the LPR by incorporating functional lipids such as DSPE-PEG-Maleimide, DSPE-PEG-NHS, DSPE-PEG-SH, DSPE-PEG-COOH or other hydrophilic phospholipids linked with -Maleimide/—NHS/—SH/—COOH functional groups that can be further conjugated to targeting molecules such as antibodies and peptide ligands. As an example, DSPE-PEG is replaced with DSPE-PEG-X where X=maleimide, or NHS or SH or COOH in the post insertion step. Where targeting molecules are required, the targeting molecules (antibodies, peptides, proteins) are added into the LPR solution and allowed to mix overnight in order for the conjugation reaction to proceed to completion. After completing the conjugation reaction, the unreacted functional groups are quenched followed by dialysis to purify the LPR.

DSPE-PEG itself can not react with antibodies, peptides and proteins. Functionalized hydrophilic phospholipids such as DMG-PEG-X and DSPE-PEG (-maleimide or —NHS or —SH or —COOH) must be used. Which functionalized DSPE-PEG to use depends on the detailed chemical conjugation reactions

The invention contemplates introduction into cells of a recombinant nucleic acid useful in many applications including, but not limited to, gene therapy, treatment for viral diseases, and RNA vaccination.

### Example—Protocol for Synthesizing LPR

STEP 1a—Preparation of Cationic Liposomes

Cationic liposomes are prepared using thin film hydration method. A 1:1 molar ratio combination of DOTAP and cholesterol dissolved in chloroform are vacuum dried. The resulting thin lipid film is then hydrated in 50° C. distilled water by vortexing for 1 min followed by sonicating in 50° C. water bath for 5˜10 min. The final total lipid concentration is adjusted to be 20 mM.

STEP 1b—Preparation of Anionic Liposomes

Anionic liposomes are prepared using thin film hydration method. A 2:1:1 molar ratio combination of DOPA, DOPE, and cholesterol respectively dissolved in chloroform are vacuum dried. The resulting thin lipid film is then hydrated in 50° C. distilled water by vortexing for 1 min followed by sonicating in 50° C. water bath for 5˜10 min. The final total lipid concentration is adjusted to be 20 mM.

STEP 2—Preparation of the Condensed Core

Next, the condensed core is prepared. In the preferred embodiment, a 4:3 weight ratio of RNA replicon to protamine is employed to prepare the Condensed Core. Accordingly, in one embodiment, 4 μg of alphavirus RNA replicon dissolved in water is mixed with 3 μg of Protamine by pipetting, and letting the combination rest at room temperature for 10 min. The claimed method has been used with an RNA replicon encoding non-structure proteins, and luciferase as the gene of interest. The mole amount of each reagent will be proportionally increased or decreased when formulating different doses of RNA replicons.

STEP 3—Creation of Final LPR

To prepare the nanoparticle for transfection, 19.36 μL of the 20 mM cationic liposome is added to the condensed core mixture of Step 2 and mixed well. The nanoparticle-Condensed Core solution is allowed to rest at room temperature for 10 min. Following this 10 minute incubation, the aforementioned solution is coated with a hydrophilic polymer layer. In the preferred embodiment, the solution is DSPE-PEG dissolved in water, and placed at room temperature (or alternatively heated at 50° C. on a hot plate for 10˜15 min). The LPR is now ready for transfection. For replicon RNA, the amount of DSPE-PEG added in the mixture is 1%˜5% of the total lipids of LPR. For other types of nucleic acid, the amount of DSPE-PEG can be adjusted accordingly. In general the density of DSPE-PEG can be 10˜20% of the total lipids in LPR.

STEP 4—Transfection Using LPR

For the purposes of illustration, using C2C12 myotubes, 24-well plates are coated with gelatin using approximately 250 mL of 2% cell culture grade gelatin. After roughly five minutes, the gelatin is aspirated out. Approximately 150,000 C2C12 cells suspended in DMEM+10% FBS are added to each well (or alternatively, fewer cells are seeded and they are allowed to grow within the cells for roughly two days depending on the rate of growth). When the cell population is confluent, the media is replaced with DMEM+2% horse serum. Fresh media is replenished each day until the cells have stopped changing morphologically. When this occurs, the media is returned to DMEM+10% FBS to nourish the cells during transfection. To transfect cells, the resulting LPR is incubated with mammalian cells for 12 h˜48 h at 37° C. in an atmosphere of 5% carbon dioxide. For other cells, the standard cell culture protocols will be used as suggested by the American Type Culture Collection (ATCC).

After the transfection period, the cells are harvested for proper analysis.

### DRAWINGS AND EXPERIMENTAL DATA

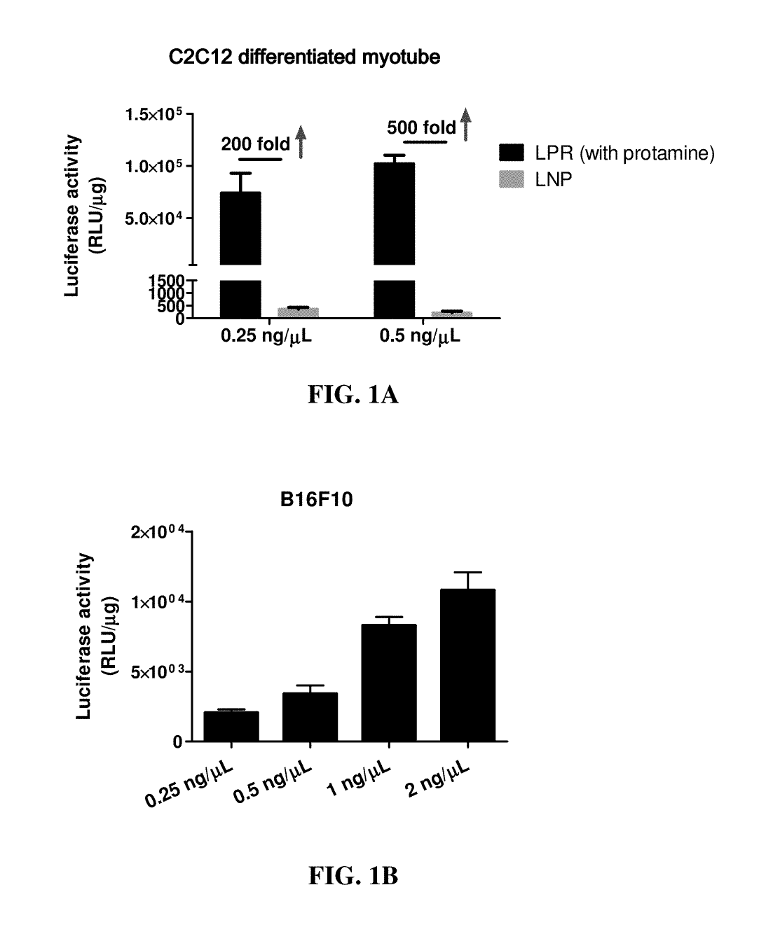

FIGS. 1A-1C demonstrate transfection efficacy of RNA replicon encoding luciferase reporter gene in various cell lines. Different concentrations of RNA replicon encoding a luciferase gene incorporated into LPR nanoparticles formulated with Protamine are transfected into mammalian cells for 24 hours. As a control, a LPR nanoparticle without the incorporation of the tested cationic peptide (in this case Protamine) was used as a control (hereinafter “LNP”). FIG. 1A shows that there is between a 200-500 fold increase in transfection efficacy employing the claimed method in the C2C12 myotubes. FIG. 1B and FIG. 1C demonstrate that the transfection efficiencies of LPR-formulated RNA replicon in B16F10 melanoma cell line and 4T1 breast cancer cell line, respectively. Further, FIGS. 1A-1C reveal that there is a dose-dependent response between dose of LPR-formulated RNA replicon and the expression level of the encoded gene. In general, across different cell lines, increasing the amount of cationic peptide and its complexed RNA replicon in cell culture media improves the transfection efficacy.

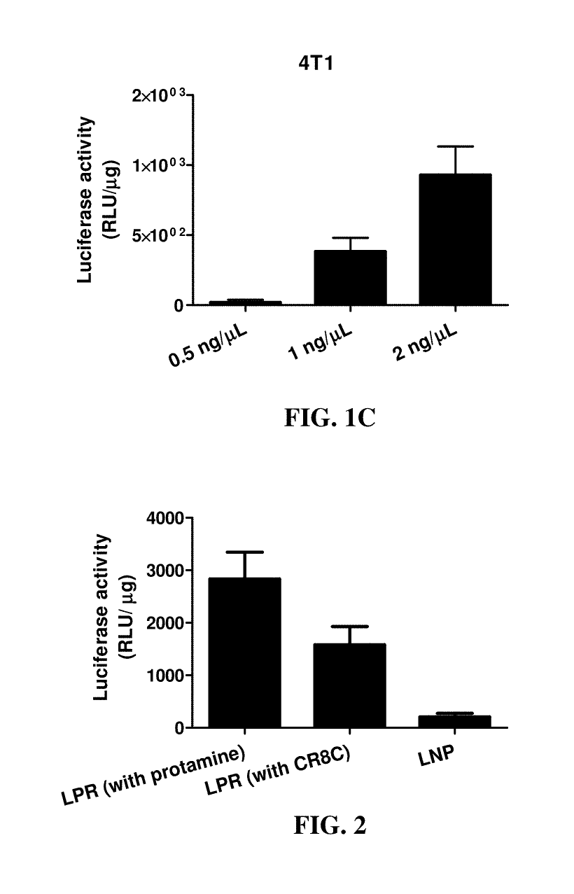

FIG. 2 demonstrates relative transfection efficacy with either Protamine LPR or CR8C LPR in B16F10 melanoma cells. The data suggests that all in all, incorporating cationic peptide to nucleic acid-loaded lipid nanoparticles increases the rate of transfection with Protamine peptide resulting in greater transfection rate than the CR8C peptide in B16F10 cells.

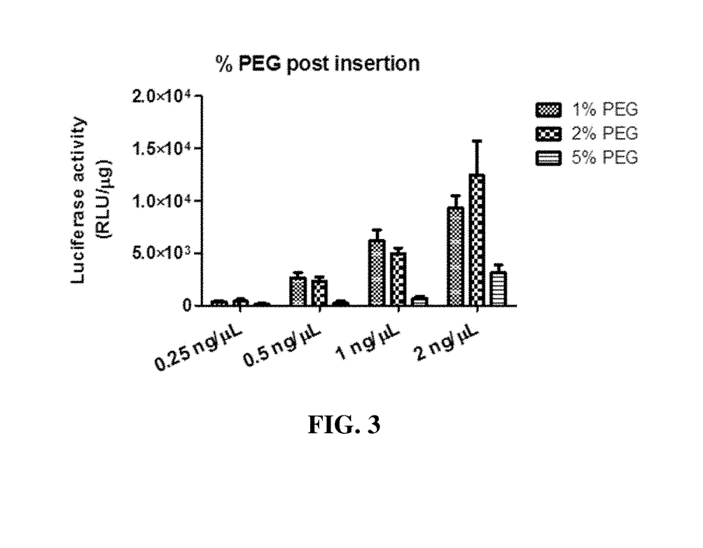

FIG. 3 demonstrates that one factor that contributes to the increased efficacy of transfection pursuant to the current protocol is the relative amount of DSPE-PEG (abbreviated as “PEG” in this figure) compared to the total amount of lipids. In general, coating the nanoparticle as described in Step 3 herein with 1%-2% DSPE-PEG (relative to the amount of total lipids in the nanoparticle) yields higher transfection efficacy for the delivery of RNA replicon.

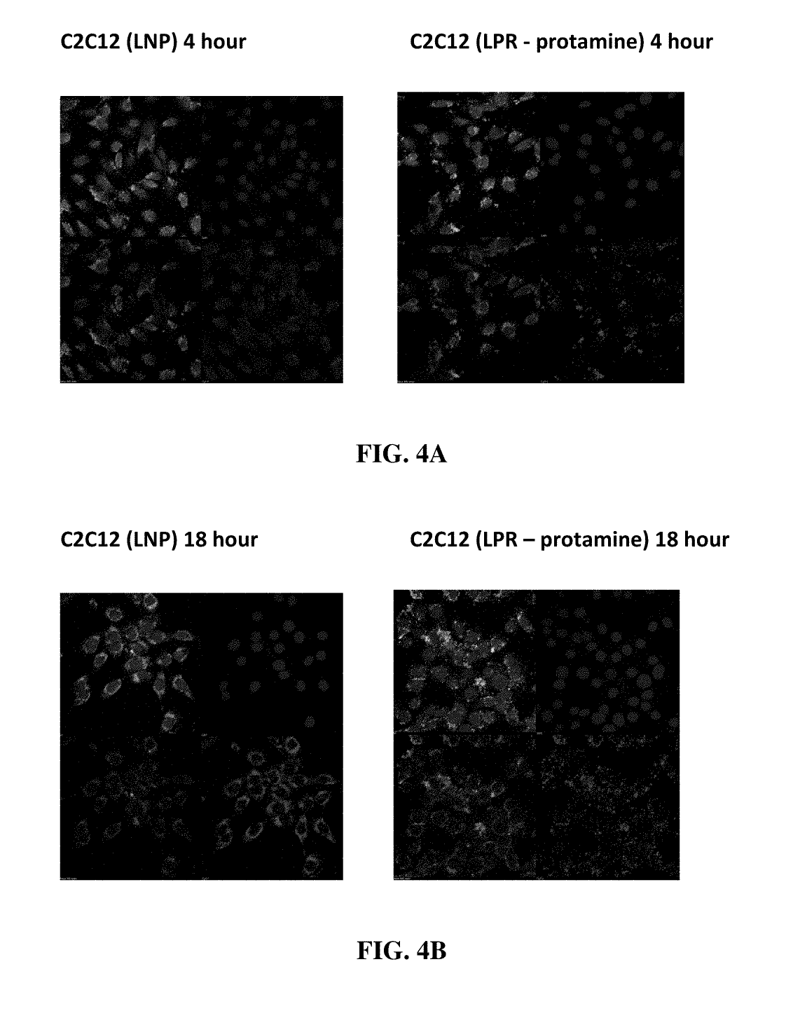

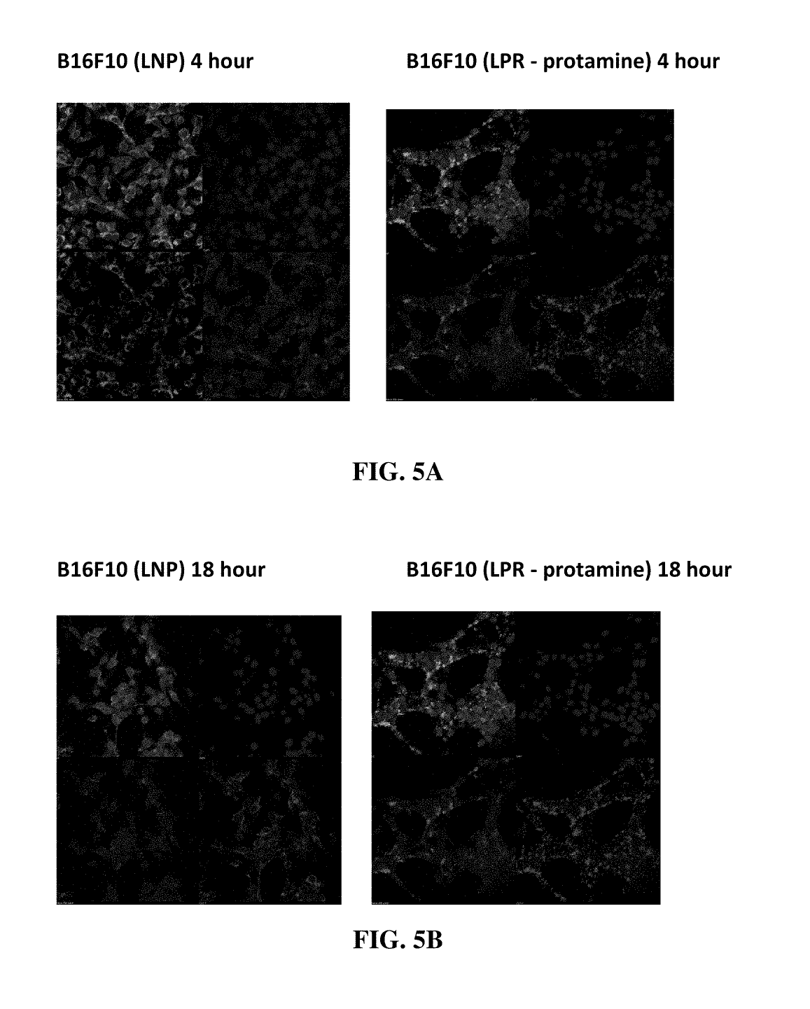

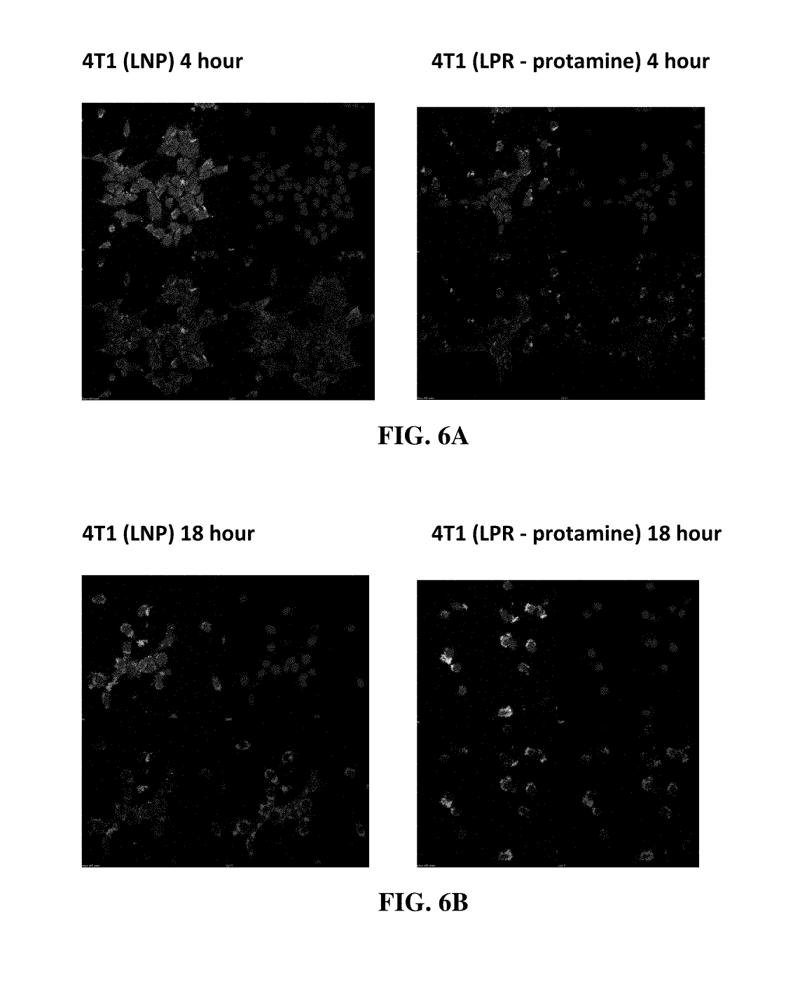

It is not yet clear as to why the incorporation of a cationic peptide within a nanoparticle improves transfection efficacy. It has been postulated that the increase may be due to enhanced endosome/lysosome escape capacity of the LPR upon entry into the cell. However, experimental data observing the intracellular distribution of nucleic acid after transfection reveals that the control LNP expresses more uniform release of the exogenous RNA replicon than the peptide containing LPR. As examples, consider the data presented in FIGS. 4A-6B. These data display the intracellular distribution of the control LNP and the peptide-containing LPR in different cell lines and as viewed through a confocal microscope. The area appearing as blue represents the cell nucleus (as stained by DAPI); the area in green represents lysosomes (as stained by Lysotracker Green DND-26); and the area in red shows the distribution of the nucleic acid (in this case, alphavirus RNA replicon encoding luciferase gene and labeled with Cy3). The data suggests that despite increases in transfection efficiency, even after eighteen hours of transfection, there is limited cytosolic release of the nucleic acid in LPR. This implies that the Condensed Core is still tightly associated with the nanoparticle whereas conventional wisdom would suggest the opposite results. Accordingly, the mechanism of the enhanced transfection by LPR remains to be answered.

In general, the invention is directed to the transfection of an alphavirus RNA replicon, but virtually any species of nucleic acid may be used, including but not limited to DNA, RNA, siRNA, mRNA or PNA. The nucleic acid can be of any length and can be single stranded, double stranded, triple stranded, cyclic nucleotide or mixtures thereof. The nucleic acids may also be modified (e.g. by epigenetic modification) or substituted using synthetic analogs.

Different types of nucleic acids may need substituted or additional components to maximize the transfection efficiency. For example, if the Condensed Core consists of small nucleic acids (e.g. siRNAs), it may be necessary, depending on the nucleic acid sequence, to further add high molecular weight carrier molecules within the Condensed Core to serve as a carrier for the small nucleic acids. Examples of such high molecular weight carrier molecules include, but are not limited to DNA (i.e. calf thymus DNA) and polysaccharides (i.e. heparin, hyaluronic acid, and their derivatives).

While multiple embodiments are disclosed, still other embodiments of the present invention will become apparent to those skilled in the art from this detailed description. There may be aspects of this invention that may be practiced without the implementation of some features as they are described. It should be understood that some details have not been described in detail in order to not unnecessarily obscure the focus of the invention. The invention is capable of myriad modifications in various obvious aspects, all without departing from the spirit and scope of the present invention. Accordingly, the drawings and descriptions are to be regarded as illustrative rather than restrictive in nature.

1. A method for administering genetic materials into eukaryotic cells comprising of:
preparing a condensed core comprising of nucleic acids and peptides;
combining said condensed core with liposomes to form a nanoparticle; and
transfecting said nanoparticle into eukaryotic cells.

2. The method of claim 1, wherein said peptides are cationic peptides.

3. The method of claim 2 wherein said cationic peptide comprises of arginine residues.

4. The method of claim 2, wherein said cationic peptide is a protamine peptide.

5. The method of claim 1, wherein the peptides are water soluble.

6. The method of claim 1, wherein said peptides are anionic peptides.

7. The method of claim 1, wherein said liposome is a cationic liposome comprised of cationic lipids.

8. The method of claim 7, wherein said cationic lipids comprise of at least one amine group.

9. The method of claim 7, wherein said cationic liposome and condensed core mixture is coated with hydrophilic phospholipids.

10. The method of claim 7 wherein said cationic liposomes are comprised of a 1:1 molar ratio of 1,2-dioleoyl-3-trimethylammonium-propane (DOTAP) and cholesterol.

11. The method of claim 1, wherein said liposome is an anionic liposome comprised of anionic lipids.

12. The method of claim 11 wherein said anionic liposomes are composed of a 2:1:1 molar ratio of 1 2-dioleoyl-sn-glycero-3-phosphate (DOPA), 1 2-dioleoyl-sn-glycero-3-phosphoethanolamine (DOPE), and cholesterol.

13. The method of claim 1, wherein the combination of condensed core and liposomes is further combined with hydrophilic phospholipids in a density less than 20% of the total lipids of the nanoparticle.

14. The method of claim 1, wherein said condensed core is negatively charged.

15. The method of claim 1, wherein said condensed core is positively charged.

16. The method of claim 1, wherein said nucleic acid comprises of small nucleic acids that weigh less than 100,000 Da.

17. The method of claim 16, wherein the condensed core is further combined with high molecular weight carrier molecules.

18. The method of claim 16, wherein the condensed core is further combined with polysaccharides or high molecular weight DNA.

19. The method of claim 1 wherein such genetic materials is administered in vitro, in vivo, or ex vivo.

20. A method for administering genetic materials into eukaryotic cells comprising of:
preparing a condensed core comprising of RNA replicon and cationic peptides;
combining said condensed core with liposomes comprised of cationic lipids to form a nanoparticle,
adding 1,2-distearoyl-sn-glycero-3-phosphoethanolamine-N-[methoxy(polyethylene glycol)-2000 (DSPE-PEG) to said mixture of condensed core and liposomes; and
transfecting said condensed core/liposome/DSPE-PEG mixture into eukaryotic cells.

21. The method of claim 20, wherein the density of DSPE-PEG is less than 5% of the total lipids of the nanoparticle.
